(19) United States
(12) Patent Application Publication
(10) Pub. No.: US 2009/0187111 A1
(43) Pub. Date: Jul. 23, 2009

(54) GAS ANALYZER

(75) Inventors: Kevin John Reilly, JR., Ridgewood, NJ (US); Mario Suttora, East Rutherford, NJ (US)

Correspondence Address: HOFFMANN & BARON, LLP, 6900 JERICHO TURNPIKE, SYOSSET, NY, 11791, US

(73) Assignee: FSP INSTRUMENTS, INC., Hoboken, NJ (US)

(21) Appl. No.: 12/301,254

(22) PCT Filed: Mar. 10, 2008
(86) PCT No.: PCT/US08/56387

§ 371 Date: Nov. 18, 2008

Related U.S. Application Data
(60) Provisional application No. 60/893,685, filed on Mar. 8, 2007.

(51) Int. Cl. A61B 5/097; G01N 1/22

(52) U.S. Cl. 600/532; 422/84

(57) ABSTRACT

The subject invention is directed to a breath analyzer which is capable of detecting toxic gas levels from breath analysis. The subject invention includes a mouthpiece which is in communication with a plurality of discrete chambers, such as first and second discrete chambers, each being provided with a separate probe for breath analysis. The probes are connected to analyzers for determining detected levels of gas. In a first embodiment, a first probe may be provided for carbon monoxide detection with a second probe being provided for hydrogen cyanide detection. Advantageously, with this arrangement, breath analysis may be conducted on-site, for example at the site of a fire, to quickly and simultaneously determine carbon monoxide and hydrogen cyanide levels in a person's blood stream. In a second embodiment, a first probe may be provided for detection of carbon monoxide and a second probe may be provided for detection of hydrogen. With this arrangement, a calibrated correction of measured carbon monoxide data can be made to correct for improperly detected hydrogen. As such, a highly accurate on-site measurement for carbon monoxide can be achieved.

### CROSS-REFERENCE TO RELATED APPLICATION

This application claims priority of U.S. Provisional Patent Application No. 60/893,685, filed on Mar. 8, 2007, the entire contents of which are hereby incorporated by reference.

### BACKGROUND OF THE INVENTION

Gas analyzers, particularly breath analyzers, are known in the prior art for detecting levels of toxins or other undesired substances in a person's body based on analysis of a person's expelled breath. A common form of breath analyzer is an alcohol breath analyzer which detects the level of alcohol in a person's blood stream based on measurements taken from the person's breath. Other forms of detectors are also known.

Carbon monoxide (CO) poisoning is common amongst individuals exposed to smoke, particularly fire victims and firefighters. Studies have found that levels of carbon monoxide in a person's blood stream can be detected by breath analysis. Such tests are typically done in a clinical or laboratory setting with results not being obtainable instantaneously.

Hydrogen cyanide (HCN) is a toxic gas which is generated through combustion of certain organic and synthetic materials. Individuals exposed to smoke are at risk of being poisoned with hydrogen cyanide. It has been found that breath analysis may provide an indication of hydrogen cyanide levels in a person's blood stream. See, e.g., U.S. Pat. No. 5,961,469 to Roizen et al., Col. 7-Col. 8.

### SUMMARY OF THE INVENTION

The subject invention is directed to a breath analyzer which is capable of detecting toxic gas levels from breath analysis. The subject invention includes a mouthpiece which is in communication with a plurality of discrete chambers, such as first and second discrete chambers, each being provided with a separate probe for breath analysis. The probes are connected to analyzers for determining detected levels of gas. In a first embodiment, a first probe may be provided for carbon monoxide detection with a second probe being provided for hydrogen cyanide detection. Advantageously, with this arrangement, breath analysis may be conducted on-site, for example at the site of a fire, to quickly and simultaneously determine carbon monoxide and hydrogen cyanide levels in a person's blood stream.

In a second embodiment, a first probe may be provided for detection of carbon monoxide and a second probe may be provided for detection of hydrogen. With this arrangement, a calibrated correction of measured carbon monoxide data can be made to correct for improperly detected hydrogen. As such, a highly accurate on-site measurement for carbon monoxide can be achieved.

These and other features of the invention will be better understood through a study of the following detailed description and accompanying drawings.

### BRIEF DESCRIPTION OF THE DRAWINGS

FIG. 1 is a perspective view of a breath analyzer formed in accordance with the subject invention;

FIG. 2 is a plan view of two chambers useable with the subject invention;

FIG. 3 is a schematic of two chambers useable with the subject invention;

FIG. 4 is a schematic of three chambers useable with the subject invention;

FIG. 5 is a schematic of an electronic configuration useable with the subject invention; and,

FIG. 6 is a schematic of a possible display arrangement useable with the subject invention.

### DETAILED DESCRIPTION OF THE INVENTION

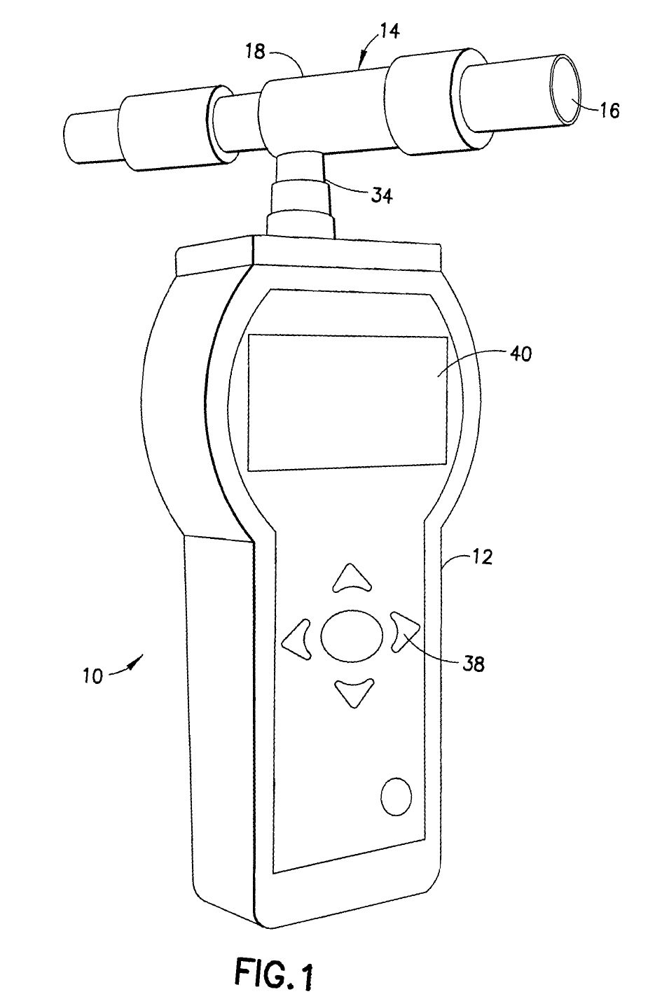

A breath analyzer 10 is provided herein which generally includes a housing 12 operatively coupled to a breath passage 14. The breath passage 14 includes a mouthpiece 16 which is open and formed to be comfortably accommodated by the mouth of a user. To use the breath analyzer 10, a user blows into the mouthpiece 16 of the breath passage 14. As shown in FIG. 1, the breath passage 14 may be a separate component from the housing 12 and be coupled thereto. Alternatively, the breath passage 14 may be disposed within the housing 12. It is preferred that the breath analyzer 10 be portable and be hand-held.

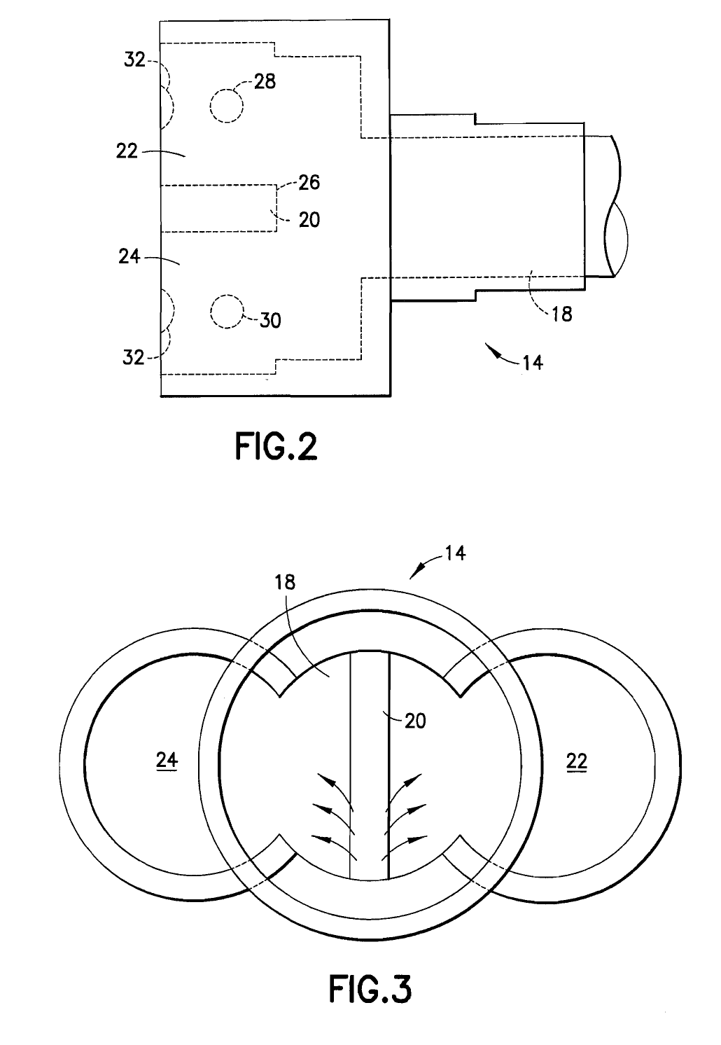

With reference to FIG. 2, the breath passage 14 includes a channel 18 that extends from the mouthpiece 16 and terminates at divider 20. The mouthpiece 16 may be a “drool-free” mouthpiece to minimize delivery of saliva into the channel 18. In addition, the mouthpiece 16 may be formed removable and replaceable for hygienic considerations. Single use of the mouthpiece 16 is preferred, although the mouthpiece 16 may be sterilized or otherwise cleaned between users.

The divider 20 is situated in the breath passage 14 to define at least first and second discrete chambers 22, 24. The first and second chambers 22, 24 can be formed with various configurations, but are preferably elongated (e.g., cylindrical) to provide an unobstructed flow path for entrapped breath. The chambers 22, 24 may be arranged parallel and may be arranged to be generally side-by-side. It is preferred that the divider 20 be located to divide breath directed down the channel 18 into equal portions into the first and second chambers 22, 24. With reference to FIG. 3, it is preferred that the divider 20 be located centrally relative to the channel 18. As represented by the arrows in FIG. 3, breath delivered down the channel 18 is diverted into the first and second chambers 22, 24. As will be appreciated by those skilled in the art, and as discussed below, additional chambers may be provided, with the divider 20 being preferably formed centrally to direct equal amounts of delivered breath to the chambers.

The divider 20 is formed with a leading edge 26 shown to be a flat surface disposed generally perpendicularly to the longitudinal axis of the channel 18. The leading edge 26 can be formed with various configurations, such as being wedge shaped or rounded to provide minimal backward deflection of delivered breath (i.e., deflection back towards the channel 18).

The first chamber 22 is provided with a first probe 28 while the second chamber 24 is provided with a second probe 30. Any probe known in the art for detecting gas levels is usable with the subject invention. To ensure movement of delivered breath across the respective probe 28, 30, a vent 32 may be provided at the rear portion of each of the first and second chambers 22, 24. With this arrangement, an unobstructed air flow from the channel 18, through the first and second chambers 22, 24, and across the first and second probes 28, 30 may be achieved.

The first and second probes 28, 30 may be selected to detect simultaneously two different types of gas. In a preferred arrangement, the first probe 28 may be a carbon monoxide probe, while the second probe 30 may be a hydrogen cyanide probe. Carbon monoxide probes are known in the prior art and may be selected from electrochemical, infrared and semiconductor-base probes, although electrochemical probes are preferred herein. In addition, it is preferred that the carbon monoxide probes be three-electrode probes and that the probes be capable of detecting 0-500 (parts per million (ppm)), more preferably 0-200 ppm, of carbon monoxide. It is preferred that the carbon monoxide probe have a high resolution over the entire detection range, preferably a resolution of 1 ppm increments.

Hydrogen cyanide probes are known in the prior art and have been used in various industries, including the electroplating industry, and may be selected from electrochemical, infrared and semi-conductor base probes, preferably electrochemical probes. It is also preferred that the probes be three-electrode probes and that the probes be capable of detecting 0-50 (parts per million (ppm)), more preferably 0-30 ppm, of hydrogen cyanide. It is preferred that the hydrogen cyanide probe have a high resolution over the entire detection range, preferably a resolution of 200 (parts per billion (ppb)) increments.

Any probes selected for use with the breath analyzer 10 are preferably probes which detect a level of a target gas and produce a corresponding electrical signal which may be processed. Probes capable of detecting other toxic, gases may also be utilized.

In a second arrangement, the first probe 28 may be a carbon monoxide probe with the second probe 30 being a hydrogen probe. Any known hydrogen probe may be utilized. With this arrangement, the second probe 30 may be used to detect hydrogen levels in the delivered breath. Carbon monoxide probes may have cross-sensitivity to hydrogen and improperly detect hydrogen along with carbon monoxide in providing errant readings. This is a particular concern with lactose-intolerant individuals who expel higher than normal levels of hydrogen. The detected levels of carbon monoxide by the first probe 28 may be corrected to take into account the actual detected hydrogen levels. In particular, hydrogen may cause a 5%-30% error in the carbon monoxide reading. Thus, it is preferred that a hydrogen correction factor be determined by calculating a predetermined value in the range of 5%-30%, more preferably in the range of 10%-12%, of the detected hydrogen level. For example, with a 10% correction factor, a hydrogen correction factor is determined by multiplying 0.10 times the detected hydrogen level. The determined hydrogen correction factor is then subtracted from the detected carbon monoxide level to obtain a corrected carbon monoxide level. The corrected level is taken as the actual detected level. The actual correction factor may be determined during calibration of the analyzer 10. A more accurate carbon monoxide measurement may be obtained with the simultaneous use of the first and second probes 28, 30.

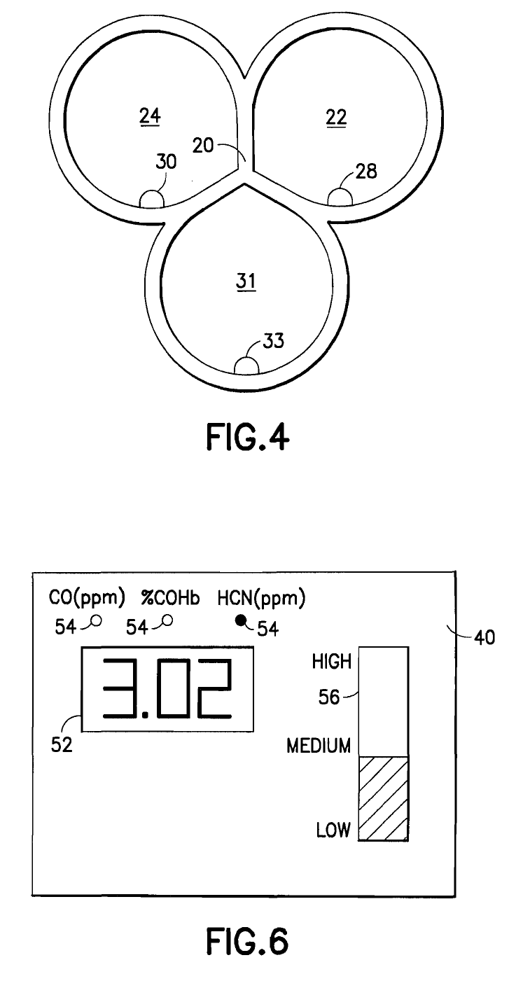

With reference to FIG. 4, a third chamber 31 may be provided, formed in similar manner to the first and second chambers 22, 24. The third chamber 31 is preferably elongated (e.g., cylindrical); arranged parallel to one or both of the first and second chambers 22, 24; and, arranged side-by-side to one or both of the first and second chambers 22, 24. The third chamber 31 may be also provided with a vent. It is preferred that the divider 20 be arranged centrally to generally direct equal amounts of breath into each of the three chambers 28, 30, 31. A third probe 33 may be provided in the third chamber 31, e.g., to permit simultaneous detection of carbon monoxide, hydrogen and hydrogen cyanide.

With reference to FIG. 1, the breath passage 14 may be rigidly fixed to the housing 12 by connector 34. Any mode of forming a connection is useable with the subject invention.

The housing 12 accommodates circuitry and power supply to collect data from the first, second and third probes 28, 30, 33 and to calculate the detected levels of gas. The first, second and third probes 28, 30, 33 are electrically coupled to the circuitry within the housing 12 preferably through the connector 34 which is hollow. As will be appreciated by those skilled in the art, any type of circuitry which is capable of manipulating the detected data is usable with the subject invention. A display 40 is provided to display the detected levels of gas.

To permit use of the breath analyzer 10 on-site at hazardous locations, particularly at the site of a fire, the housing 12 is preferably formed of robust and durable materials which protect the contained circuitry from water damage, heat and other hazardous conditions. In addition, the breath passage 14, the mouthpiece 16 and the connector 34 are formed from robust materials to also withstand such conditions. It is preferred that the mouthpiece 16 be formed from a durable plastic material to be more comfortably used. The mouthpiece 16 may be formed of an acetal resin, such as that sold under the trademark “DELRIN” by DuPont Corporation.

By way of non-limiting example, and with reference to FIG. 5, the housing 14 may accommodate a microprocessor, microcontroller or any other CPU variant 42. The microprocessor 42 may be electrically coupled to the first, second and third probes 28, 30, 33 via amplifiers 44 (e.g., high-precision amplifiers). Low-level current signals generated by the probes 28, 30, 33 (e.g., on a nano-amp range) in response to gas detection may be converted to working voltage levels by the amplifiers 44. The converted analog voltage levels are further processed by analog-to-digital converters (ADC) 45 to produce digital signals which may be manipulated by the microprocessor 42. The signal from each of the probes 28, 30, 33 is preferably separately processed. Connections between the probes 28, 30, 33 and the microprocessor 42 are preferably assembled to be hidden from ambient exposure, for example, in the breath passage 14 and the connector 34.

The microprocessor 42 is configured to obtain raw data from the probes 28, 30, 33 and to evaluate blood stream gas levels from the raw data. The breath analyzer 10 may also be provided with an electronic storage or memory 36 to record obtained data (raw data as measured by the probes and/or data which has been calculated by the microprocessor 42). The memory 36 may be a memory chip, such as an EPROM or flash memory. It is preferred that obtained data alone not be stored, but be stored along with a time and date stamp. As such, a timer 46 is also preferably included with the breath analyzer 10. Other identifiers may be saved with the obtained data. To permit inputting of other identifiers, an input device 38, such as a key pad, track pad, and/or buttons, may be mounted onto the housing 12. Through coordination of the input device 38 and the display 40, identifying information such as name, weight, height, age, sex, medical conditions, health conditions (e.g., smoker vs. non-smoker), or alerts (e.g., allergies) may be inputted into the breath analyzer 10 for association, and storage, with the corresponding obtained data.

The probes 28, 30, 33, depending on their configuration, may be continuously activated (i.e., continuously detecting) or may be selectively activatable (e.g., activated to an activation state for monitoring). In either regard, the probes 28, 30, 33 need to be fully activated to operate properly for detection. With full activation, the probes 28, 30, 33 may be brought to a “ready” state where the output signals of the probes 28, 30, 33 may be transmitted to the microprocessor 42, as discussed above. In a non-ready state, the output signals need not be transmitted to the microprocessor 42 (thus possibly saving power). The input device 38 may be configured to activate a ready state for the analyzer 10.

Prior to, or once, ready, it is preferred that the analyzer 10 conduct a baseline test to evaluate ambient conditions. The baseline test is conducted with the mouthpiece 16 open and unobstructed. Ambient conditions of the analyzer 10 may include toxic gas. For the baseline test, the probes 28, 30, 33 detect levels of ambient gas, and these levels are stored in the memory 36. Thereafter, the analyzer 10 is readied for actual testing, and actual testing is conducted, as described below, with the probes 28, 30, 33 detecting gas levels in a person's expelled breath. The detections by the probes 28, 30, 33 may be conducted over predetermined intervals of time, e.g. determined by the timer 46. Alternatively, or in addition, a stop signal may be manually entered. In this manner, start and stop of a detection cycle may be defined. The highest readings detected by the probes 28, 30, 33 during a testing interval (ambient or actual) are taken as the detected levels. The baseline results may be utilized to adjust the actual obtained results to correct for ambient conditions. The baseline results may be directly subtracted from the actual results or the baseline results may be applied to the actual results in the same manner as the detected hydrogen levels are applied to the carbon monoxide levels for correction, as described above. The application of the baseline results may be determined during calibration of the analyzer 10.

As is known in the prior art, the microprocessor 42 may be electrically coupled to the probes 28, 30, 33; the memory 36; the input device 38; the display 40; and, the timer 46. The microprocessor 42 may be formed to control and coordinate all of these elements, as is known in the prior art. In addition, a power supply 48 is provided which is preferably rechargeable, such as a lithium-ion cell. Any known mechanism for activating and deactivating electronic circuitry may be utilized with the subject invention.

To permit access to the stored data, any known technology or technique may be utilized. For example, a port 50, such as a USB port, may be provided to permit a hard-wire connection to the breath analyzer 10 for downloading of collected information. Other means, such as an infrared transmitter/receiver or wireless transmitter/receiver may also be utilized.

Test results provided by the probes 28, 30, 33 and obtained by the microprocessor 42 may require conversion or other manipulation to appreciate a dangerous blood level content. For example, a detected carbon monoxide level requires manipulation to produce a percent carboxyhemoglobin (% COfb) number which is an indication of a person's state of carbon monoxide level in his hemoglobin. Carbon monoxide can cause hemoglobin to convert to carboxyhemoglobin; carboxyhemoglobin prevents the associated hemoglobin from delivering oxygen to various areas of the body. Excessive carboxyhemoglobin may result in dangerous levels of oxygen deprivation. To obtain a carboxyhemoglobin percentage, the actual detected carbon monoxide (CO) level (detected in units of parts per million (ppm)) is mathematically manipulated as follows: % COHb=(0.16×(CO ppm))+0.5. The calculated % COHb may be displayed on the display 40. As recognized by those skilled in the art, any % COHb number above 10% may be symptomatic, whereas, even 5% may be an indication of danger. If desired, the actual measured CO level (ppm) may be displayed on the display 40. Both the measured CO level (ppm) and the carboxyhemoglobin level (% COHb) may be stored in the memory 36 for later analysis.

If hydrogen levels are measured, the detected carbon monoxide levels may be corrected, as described above, prior to calculation of carboxyhemoglobin levels. The un-corrected and corrected CO levels may be saved along with the % COHb.

With respect to the detection of hydrogen cyanide, a direct correlation between a blood stream level and breath content has not been determined. However, hydrogen cyanide is foreign to the body, and its presence in the body indicates some level of toxicity. It is possible to display on the display 40 the actual detected level of hydrogen cyanide (parts per million (ppm)). The actual detected level will provide medical or emergency personnel with an indication of the possible level of hydrogen cyanide poisoning. Emergency treatment may be determined based on the evaluation of the actual detected level.

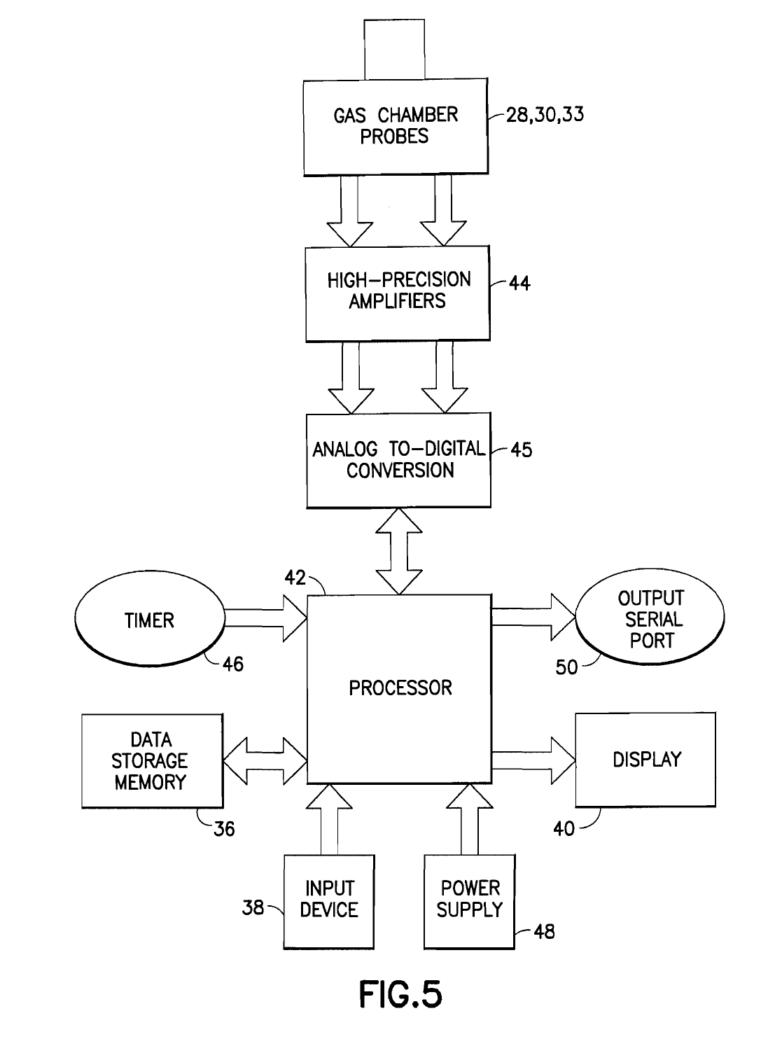

As shown in FIG. 6, the display 40 may include one or more numeric fields 52 for displaying numeric values. Indicators 54 may be provided to indicate the measured item (e.g., CO level; HCN level; % COHb) corresponding to the displayed numeric value in one or one of the numeric fields 52. There can be a one-to-one correspondence of the numeric fields 52 to the various items being evaluated by the breath analyzer 10 (e.g., three possible outputs (CO level; HCN level; % COHb) equal three numeric fields). A less than one-to-one correspondence can be utilized with the indicators 54 being provided as needed. It is noted that the displayed numeric amount can be evaluated outside of the breath analyzer 10 . . . . For example, a user may have a chart or other guide which correlates a displayed amount to a convertible standard (e.g., for detecting toxic levels).

In addition to, or as an alternative, one or more graphic representations 56 may be utilized to graphically indicate the measured level of a particular gas. The graphic representations 56 may provide graphically general areas of possible results (e.g., High Risk; Medium Risk; Low Risk) with an indication of where actual detected levels fall. By way of non-limiting examples, the graphic representation 56 may be a bar or linear graph, a wheel, a needle gauge, or combinations thereof. All or portions of the graphic representations 56 may be colored, particularly to indicate different levels of concern (e.g., green to indicate safe level and red to indicate dangerous level). As with the numeric fields 52, any quantity of the graphic representations 56 may be utilized, and the graphic representations 56 may be used in conjunction with the indicators 54.

The following is an exemplary manner of operating the breath analyzer 10 (having the configuration of a carbon monoxide probe and a hydrogen cyanide probe):
activate the breath analyzer 10 and permit the device to come to a fully activated state (i.e., permit the breath analyzer 10 to fully warm up);
the breath analyzer 10 automatically conducts an ambient reading to determine baseline measurements of gas (e.g., ambient levels of carbon monoxide and hydrogen cyanide will be determined);
patient data may be inputted;
instruct patient to take and hold a deep breath for approximately 15 seconds prior to testing;
the breath analyzer 10 is activated to a ready state and the patient exhales into the mouthpiece 16 of the breath passage 14 with the patient's full tidal breath being captured within the breath passage 14;
the breath analyzer 10 determines the detected levels of gas; the detected levels may be adjusted for the pre-determined baseline measurements (e.g., the baseline measurements may be subtracted from the detected levels); and,
the mouthpiece 16 may be replaced, wiped or sterilized prior to a next patient using the breath analyzer 10.
   Other configurations of the breath analyzer 10 may operate in similar fashion.

Over a course of repeated tests, the breath analyzer 10 may be configured to re-test ambient conditions to re-set the baseline measurements. Ambient testing can be conducted before each patient test. Also, the breath analyzer 10 may be configured to test from zero and over a range. Alternatively, the analyzer 10 may be configured with minimum threshold levels so that only measurements above the threshold values will register, be displayed and/or be stored. For example, a carbon monoxide level of one part per million (ppm) and a hydrogen cyanide level of one part per billion (ppb) may be set as the minimum threshold values.

If a patient provides a test result of concern, it is recommended that an interval of time be waited and that the patient be re-tested. Repeated testing will provide an opportunity to ensure accurate detection and the possibility of identifying an actual peak reading. It is also recommended that at least 10 minutes be waited after a patient smokes before being tested to avoid false readings.

The subject invention allows for simultaneous elevation of at least two different gases from a person's expelled breath. Under emergency conditions, rapid and simultaneous recognition of poisoning may be critical to treatment. The analyzer 10 permits simultaneous evaluation of two toxic gases (e.g., CO and HCN) in a quick and efficient manner.

The breath analyzer 10 may be also utilized as a free-standing detector which measures toxic gas levels of surrounding ambient air. For example, the breath analyzer 10 may be located in or near an infant's crib to monitor toxic gas levels, particularly carbon monoxide. With this arrangement, breath is not required to be blown into the breath passage 14. Rather, testing of ambient air is conducted. It is preferred that the second arrangement discussed above, which includes the carbon monoxide probe and the hydrogen probe, be utilized as a free-standing detector to provide accurate carbon monoxide readings. The timer 46 may be configured to trigger automatic readings at fixed intervals, with such readings being recorded into the memory 36. The recorded data is then reviewable to ascertain exposure to toxic gas. Continuous monitoring is also possible with a warning signal being emitted upon sufficiently high levels of toxic gas being detected. For ambient testing, it is preferred that the probe(s) be selected to have high sensitivity and be able to detect low levels of gas, such as, for example, less than 30 parts per million (ppm) of carbon monoxide or 200 parts per billion (ppb) of hydrogen cyanide. Prior art carbon monoxide detectors are configured to detect relatively high levels of carbon monoxide. These devices have “offsets” or minimum thresholds before carbon monoxide levels are actually detected and determined. The device of the subject invention allows for not only low levels of detection without any offsets, but also detection up to zero or nil levels. These detections can be for any gas being detected, including carbon monoxide and hydrogen cyanide. Measurements of toxic gas from ambient air do not require manipulation to determine correlation to levels of the toxic gas in a person's blood stream, such as that required with breath analysis.

What is claimed is:

1. An analyzer for detecting gas levels in a person's expelled breath, said analyzer comprising:
an inlet having a first open end formed to receive a person's expelled breath;
first and second discrete chambers;
at least one channel communicating said first open end and said first and second discrete chambers; and,
means for simultaneously detecting the levels of at least two different types of gas in the expelled breath in said first and second chambers.

2. An analyzer as in claim 1, wherein said means for detecting gas levels includes means for detecting carbon monoxide levels.

3. An analyzer as in claim 2, wherein said means for detecting gas levels includes means for detecting hydrogen cyanide levels.

4. An analyzer as in claim 3, wherein said means for detecting carbon monoxide levels is adapted to detect carbon monoxide levels in said first chamber, and wherein said means for detecting hydrogen cyanide levels is adapted to detect hydrogen cyanide levels in said second chamber.

5. An analyzer as in claim 2, wherein said means for detecting gas levels includes means for detecting hydrogen levels.

6. An analyzer as in claim 5, wherein said means for detecting carbon monoxide levels is adapted to detect carbon monoxide levels in said first chamber, and wherein said means for detecting hydrogen levels is adapted to detect hydrogen levels in said second chamber.

7. An analyzer as in claim 6, wherein said means for detecting gas levels includes means for detecting hydrogen cyanide levels.

8. An analyzer as in claim 7, further comprising a third discrete chamber, and wherein said means for detecting hydrogen cyanide levels is adapted to detect hydrogen cyanide levels in said third chamber.

9. An analyzer as in claim 1, wherein said means for detecting gas levels includes means for detecting hydrogen cyanide levels.

10. An analyzer as in claim 1, wherein said first and second chambers are elongated.

11. An analyzer as in claim 1, wherein said first and second chambers are generally parallel.

12. An analyzer as in claim 1, wherein said first and second chambers are each provided with at least one vent.

13. An analyzer as in claim 1, wherein a divider is disposed between said first and second chambers to divide the expelled breath between said first and second chambers.

14. An analyzer as in claim 1, wherein said analyzer is hand-held and portable.

15. An analyzer for detecting gas levels in a person's expelled breath, said analyzer comprising:
an inlet having a first open end formed to receive a person's expelled breath;
a first chamber having a carbon monoxide probe associated therewith for detecting carbon monoxide levels in the expelled breath in said first chamber; and,
a second chamber having a hydrogen cyanide probe associated therewith for detecting hydrogen cyanide levels in the expelled breath in said second chamber.

16. An analyzer as in claim 15, further comprising a third chamber having a hydrogen probe associated therewith for detecting hydrogen levels in the expelled breath in said third chamber.

17. An analyzer for detecting gas levels, said analyzer comprising:
an inlet having an opening;
a first chamber having a carbon monoxide probe associated therewith for detecting carbon monoxide levels in said first chamber;
a second chamber having a hydrogen probe associated therewith for detecting hydrogen levels in said second chamber; and
means for adjusting said detected carbon monoxide levels in view of said detected hydrogen levels.

18. An analyzer as in claim 17, further comprising a mouthpiece formed to receive a person's expelled breath.

19. An analyzer as in claim 17, further comprising a timer, wherein said timer is configured to indicate predetermined intervals of time for detecting carbon monoxide and hydrogen levels.
